(19) United States
(12) Patent Application Publication
(10) Pub. No.: US 2016/0143576 A1
(43) Pub. Date: May 26, 2016

(54) MRI IMAGE FUSION METHODS AND USES THEREOF

(71) Applicant: TEL HASHOMER MEDICAL RESEARCH INFRASTRUCTURE AND SERVICES LTD., Ramat Gan (IL)

(72) Inventors: ZVI SYMON, RAMAT GAN (IL); ARNALDO MAYER, RAMAT GAN (IL); ADI ZHOLKOVER, KIRYAT BIALIK (IL)

(21) Appl. No.: 14/904,955

(22) PCT Filed: Jul. 14, 2014
(86) PCT No.: PCT/IL2014/050634

§ 371 Date: Jan. 18, 2016

Related U.S. Application Data
(60) Provisional application No. 61/957,868, filed on Jul. 15, 2013.

(51) Int. Cl. A61B 5/00; A61B 8/08; G01R 33/48; A61B 8/12

(52) U.S. Cl. CPC A61B 5/4381; A61B 8/12; A61B 8/085; G01R 33/4814; A61B 2576/00

(57) ABSTRACT

A method for fusing a pre-operative MRI prostate image to an intra-operative TRUS or CT prostate image according to a least-cost affine transformation of the MRI contour onto the TRUS or CT contour, with smooth non-linear warping adjustment. MRI and CT processing may be performed as a pre-operational procedure for increased efficiency, while TRUS may be performed concurrent with a surgical procedure.

### FIELD OF THE INVENTION

The present invention relates to methods generating a Magnetic Resonance Imaging (MRI)-Trans-Rectal Ultra-Sound (TRUS) Fusion image or a MRI-Computerized Tomography (CT) Fusion image of a subject's prostate for increased accuracy guiding interventions for treatment of prostate cancer or suspected prostate cancer in the subject. Further, the present invention relates to methods of use of an MRI-TRUS or MRI-CT fusion images during local therapies for treating prostate cancer or diagnosis prostate cancer.

### BACKGROUND

The treatment of prostate cancer presents a common clinical dilemma. The standard approach of curative whole gland therapy is associated with significant impact on quality of life, particularly sexual function. The alternative, active surveillance has a low, but real risk of progression and requires a combination of effective communication skills of the physician and a calm, secure and compliant patient. Recently, more accurate localization of cancers within the prostate has generated an interest in focal therapy as a less radical approach. The goal of focal therapy in prostate cancer is to achieve an optimal balance between cancer control and maintenance of quality of life.

Current interventional pre-operative Magnetic Resonance Imaging (MRI) platforms are not conducive to optimal positioning of a patient in extended lithotomy absolutely necessary for adequate transperineal approaches to all regions of the prostate gland during surgery. In contrast, intraoperative Trans-Rectal Ultrasound (TRUS) images are ideal for positioning a patient but lack details to accurately define a surgical target during prostate therapy. Alternatively, Computerized Tomography (CT) images are used in external beam radiotherapy (EBRT) of the prostate to provide radiation dose planning and image based guidance of the radiation beam with regard to the patient position. Unfortunately, neither of TRUS or CT techniques accurately detects and/or localizes tumor masses or pre-cancerous tissue within a prostate gland.

Fusion of Magnetic Resonance Imaging (MRI) with Trans-Rectal Ultra-Sound (TRUS) or Computerized Tomography (CT) provides a new approach for therapy, such as brachytherapy seed implantation, that combines the high resolution and high-contrast image quality of pre-operative MRI with the real-time interactive image guidance of TRUS during brachytherapy operative procedures or CT during EBRT procedures. Unfortunately, however, there are many factors that complicate matching MRI and, TRUS or CT images, with the result that current schemes for combining MRI and, TRUS or CT images do not attain sufficiently accurate registration to fully benefit from the combination.

A few attempts have been made to register TRUS and MRI images. The Urostan station developed by Koelis (LA Tronche, France) performs registration between TRUS and MRI images for the purpose of image guided prostate biopsies. Registration is obtained by elastic deformation of previously manually segmented prostate surfaces on MRI and TRUS scans. Operation of the Urostan station does not take into consideration information regarding the local structure surrounding the surface points but rather the spatial coordinate of the surface points. Therefore, there is no guarantee that corresponding anatomical areas or landmarks are actually mapped by the elastic deformation field.

Further, methods of use of an Urostan station incorporate only the external prostate surface in order to compute a deformation field that is extrapolated to the internal volume. Thus, this method does not incorporate information that could be provided by other anatomical structures located at the same depth of the prostate, “in-depth” structures. The consequence of not using “in-depth” structures is that the registration accuracy in the depth of the prostate is limited by the lack of structural information obtained deep inside the prostate.

In order to increase the accuracy of local therapies treating prostate cancer, there is a widely recognized need to ensure corresponding anatomical areas or landmarks are mapped and to use additional anatomical “in-depth” structures, for example the central zone, the urethra, the peripheral zone, in order to improve the registration of MRI and TRUS images.

Robotic prostatectomy, such as that performed using a DA Vinci® Robot (Intuitive Surgical), is a minimally invasive surgical method for radical prostatectomy. Optical cameras are used to provide images of the operating field but can only show the tissue layers that are not occluded by overlaying tissue layers. The situation is similar to that of a miner digging into a rock wall. Only the surface of the wall is apparent, but not what lies behind. In the case of a surgeon performing localized surgery as a treatment for prostate cancer, the surgeon cannot see behind the outermost tissue layer. There is a need to extend the surgeon's field of view beyond the “wall surface”, deep into the underlying tissue layers for increased accuracy and safety during the operation. For example, the ability to see deeper into the tissue layers could improve resection accuracy and spare the neurovascular bundle to limit iatrogenic damage to the patient.

Current technologies are lacking in accuracy for registering images of tissue structures, for example the prostrate, using different imaging technologies. Further, current technology is unable to perform neurovascular mapping, which would aid surgeons to avoid iatrogenic damages during surgeries.

Thus, there is a recognized need to provide a robust and accurate registration and fusion of images of corresponding prostate structures captured using pre-operative MRI and intraoperative TRUS images, or CT images, based on local structural information present both superficially and deep within the prostate, in order to improve the accuracy and safety of prostate surgery. In addition, there is a need to provide clinical personnel with an overlap of the mapping of important anatomical structures visible in MRI with the information in the surgical field of view (FOV), as provided by TRUS or CT, optical cameras or any other imaging method that can be aligned with MRI. There is a further need to enable focal treatment and nerve sparing in brachytherapy and robotic prostatectomy.

### SUMMARY

In one embodiment, this invention provides a method for generating a Trans-Rectal Ultra-Sound (TRUS)-Magnetic Resonance Imaging (MRI) fusion image of a prostate gland of a subject, the method comprising the following steps: (a) inputting an MRI scan of the prostate gland of the subject; (b) segmenting the MRI scan to produce at least one segmented MRI contour surface of the organ, the contour comprising a plurality of three-dimensional (3D) landmark points; (c) inputting a TRUS scan of the prostate gland of the subject; (d) segmenting the TRUS scan to produce at least one segmented TRUS contour surface of the prostate gland, the contour comprising a plurality of 3D landmark points, wherein the at least one MRI contour surface and the at least one TRUS contour surface correspond to the same anatomical surface; (e) resampling the TRUS and MRI contours to a common geometric space; (f) computing a linear transformation that maps the MRI contour surface onto the TRUS contour surface, the linear transformation being an affine transformation estimated by minimization of the matching cost between the plurality of landmark points on the MRI contour and the plurality of landmark points on the TRUS contour; (g) applying the linear transformation to the MRI contour points to obtain linearly transformed (LT) MRI contour points; (h) computing a local shape descriptor for each LT landmark point of the MRI contour surface and each landmark point of the TRUS contour surface; (i) computing an optimal assignment between the LT landmark MRI and TRUS contour surface points that minimizes a matching cost criterion between the shape descriptors of the matched points, the optimal assignment defining a sparse vector field mapping MRI contour points onto TRUS contour points; (j) computing a dense deformation field by smooth interpolation of the sparse vector field to map any point of the whole MRI volume onto a point of the TRUS volume; and (k) applying the linear and non-linear mapping of steps (f) through (j) to map points of the MRI image into the TRUS image; wherein the performance of steps (a) through (k) generates a Trans-Rectal Ultra-Sound-Magnetic Resonance Imaging fusion image of the prostate of the subject.

In another embodiment of a method of this invention the TRUS scan input is replaced by a Computerized Tomography scan image, wherein the method includes all of the same steps using the CT scan in place of the TRUS scan, and wherein the performance of steps (a) through (k) generates a Computerized Tomography-Magnetic Resonance Imaging fusion image of the prostate of the subject.

In one embodiment of a method of this invention, an at least one contour surface comprises an external surface of the prostrate or a portion thereof, a contour of an internal surface of the prostate or a portion thereof, a contour of a transitional zone of the prostate or a portion thereof, a contour of a central zone of the prostate or a portion thereof, a contour of a peripheral zone of the prostate or a portion thereof, a contour of an interface between a central zone and a peripheral zone of the prostate or a portion thereof, a contour of a surface bordering the prostate and the urethra or a portion thereof, a contour based on observable calcifications, or any combinations thereof, or any combination thereof.

In one embodiment of a method of this invention, an at least one contour surface comprises two or more contour surfaces.

In one embodiment of a method of this invention, a matching cost criterion of step (i) is computed by comparing the count distribution of contour points falling within a plurality of histogram bins neighboring each landmark point.

In one embodiment of a method of this invention, a dense deformation field is constrained to be smooth and invertible.

In one embodiment of a method of this invention, fusion images generated of the prostate gland comprises less than the complete image of the prostate gland.

In one embodiment of a method of this invention, a subject is undergoing a focal procedure.

In one embodiment of a method of this invention, a focal procedure comprises a diagnostic procedure, an intervention procedure, or a therapeutic procedure, or any combination thereof.

In one embodiment of a method of this invention, a focal procedure comprises a prostatectomy, a robotic prostatectomy, a biopsy, an image guided biopsy, brachytherapy, cryotherapy, a high intensity focalized ultrasound therapy, a vascular targeted photodynamic therapy, a radiotherapy or a surgery for removal of a tumor, or any combination thereof.

In one embodiment, a method of this invention uses a fused Trans-Rectal Ultra-Sound (TRUS)-Magnetic Resonance Imaging (MRI) image of a prostate of a subject for improving the accuracy of determining a location of target for a medical procedure, the method comprising the steps (a) through (k) recited above, wherein the performance of steps (a) through (k) generates a Trans-Rectal Ultra-Sound-Magnetic Resonance Imaging fusion image of the prostate, and wherein the fused image provides improved accuracy of determining the location of target for the medical procedure.

In another embodiment, a method of this invention the TRUS scan input is replaced by a Computerized Tomography scan image, wherein the method includes all of the same steps using the CT scan in place of the TRUS scan, and wherein the performance of steps (a) through (k) generates a Computerized Tomography-Magnetic Resonance Imaging fusion image of the prostate of the subject that provides improved accuracy of determining the location of target for the medical procedure.

In one embodiment, a method of this invention includes a medical procedure comprising a focal procedure.

In one embodiment of a method of this invention a target may be the complete prostate gland, may be a region of the prostate gland, may be a tumor within the prostate gland, or any combination thereof.

In one embodiment of a method of this invention, a subject has prostate cancer or is suspected of having prostate cancer.

In one embodiment a method of this invention is for treating or diagnosing a subject having prostate cancer, or suspected of having cancer using a fused Trans-Rectal Ultra-Sound (TRUS)-Magnetic Resonance Imaging (MRI) image of a prostate gland of the subject, the method of treatment or diagnosis comprising a surgical procedure; wherein at the time the surgical procedure the method includes the steps (a) through (k) as recited above, wherein the performance of steps (a) through (k) generates a Trans-Rectal Ultra-Sound-Magnetic Resonance Imaging fusion image of the prostate, and the fusion image is used in targeting an area of the prostate for surgical treatment or diagnosis in the subject having cancer or suspected of having cancer.

In another embodiment, a method of this invention the TRUS scan input is replaced by a Computerized Tomography scan image, wherein the method includes all of the same steps using the CT scan in place of the TRUS scan, and wherein the performance of steps (a) through (k) generates a Computerized Tomography-Magnetic Resonance Imaging fusion image of the prostate of the subject and the fusion image is used in targeting an area of the prostate for surgical treatment or diagnosis in the subject having cancer or suspected of having cancer.

In one embodiment of a method of this invention, a surgical procedure comprises a focal procedure.

In one embodiment a method of this invention generates a Computerized Tomography (CT)-Magnetic Resonance Imaging (MRI) fusion image of a prostate gland of a subject, the method comprising the steps (a) through (k) recited above.

In one embodiment, methods of this invention generating fused images provide a visualization and localization of the neurovascular bundle adjacent to the prostate gland. In one embodiment, methods of use of this invention improving the accuracy of determining the location to target during said medical procedure further comprises improving the accuracy of determining a location to avoid during the medical procedure in order that the neurovascular bundle is not damaged. In one embodiment, a method of use of this invention, damage to the neurovascular bundle is avoided during robotic prostatectomy.

### BRIEF DESCRIPTION OF THE DRAWINGS

The subject matter disclosed may best be understood by reference to the following detailed description when read with the accompanying drawings in which:

FIG. 1A presents an MRI scan image and a tracing thereof of a prostate gland, which in one embodiment may be manually drawn by a user based on a pre-operative MRI scan. FIG. 1A illustrates an embodiment of an MRI contour of a prostate gland (101, line-dot-line-dot-line shape) with a cancerous tumor (103, cross-hatch shape) visible by MRI.

FIG. 1B presents a TRUS scan image and a tracing thereof of a prostate gland, which in one embodiment may be manually drawn by a user based on an intra-operative TRUS scan. FIG. 1B illustrates an embodiment of a TRUS contour of the prostate gland (105, line-double dot-line-double dot-shape) of FIG. 1A, wherein the cancerous tumor is not visible by TRUS (107).

FIG. 1C presents the superposition of the MRI contour tracing (101) of a prostate gland showing the cancerous tumor (103) of FIG. 1A, and the TRUS contour tracing (105) of the prostate gland shown in FIG. 1B, wherein the misalignment of MRI and TRUS contours is observed. The cancerous tumor (103) presents an embodiment of a targeted focal area.

FIGS. 2A-1 and 2A-2 present one embodiment of an MRI scan (2A-1) and a tracing thereof (2A-2) of a prostate gland in two dimensions, showing representative contour points within histogram bins (◯, 209) on the MRI prostate contour (203) shown in FIG. 2A-1.

FIGS. 2B-1 and 2B-2 presents one embodiment of a TRUS scan (2B-1) and a tracing thereof (2B-2) of a prostate gland in two dimensions, showing representative contour points within histogram bins (◯, 215).

FIGS. 2C-1 and 2C-2 show the overlay of the MRI contour of FIG. 1A (225) of the prostate gland with the corresponding TRUS contour prostatic image of FIG. 1B (223) before fusion is performed. Line segments connect landmark points of the two contours that have been matched by cost minimizing optimal assignment. For example, the line segment (229) connecting a landmark point from the MRI contour (231) with landmark points from the TRUS contour (233). The cancerous tumor (227), not observable in FIG. 2C-1, is depicted in FIG. 2C-2 as at the start of the fusion process.

FIGS. 2D-1 and 2D-2 presents one embodiment of an MRI-TRUS fusion contour image (2D-1) and a tracing thereof (2D-2) of the prostate imaged in FIGS. 1A and 1B, resulting from the warping of the original MRI prostate voxels (FIGS. 2D-1 and 2D-2; 243, -------). The fused image is produced following a series of interactive selection and computational steps, one embodiment of which is schematically shown in the flow chart of FIG. 3. The location of the cancerous tumor is visualized in the MRI-TRUS fusion image (2D-1 and 2D-2) at 247 (cross-hatch), which presents a potential delimited target area for focal therapy.

FIG. 3 provides a flowchart of a method for MRI-TRUS image fusion, according to an embodiment of the present invention.

FIG. 4A provides a flowchart of a method for establishing MRI image landmarks (for example step 317 of FIG. 3), according to one embodiment of the present invention.

FIG. 4B is a flowchart of a method for MRI-TRUS image fusion, according to one embodiment of the present invention.

FIGS. 5A-D present an embodiment of a TRUS scan image (5A) of a prostate gland, wherein a cancer is not visible and the corresponding MRI scan image of the prostate gland showing the cancer (5B—cancer is encircled) and the fused MRI-TRUS image (5C) of the prostate gland that could be used by medical personnel during a surgical procedure, showing increased definition of the prostate compared to the TRUS alone and wherein the location of a target cancer is identifiable (white circle). In FIG. 5D, small boxes mark the target area for a medical treatment, wherein in certain embodiments, there is now increased accuracy of treatment, for example, for brachytherapy.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements. In addition, illustrations have been provided as two dimensional images for simplicity and clarity.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment this invention comprises, a method for generating a Trans-Rectal Ultra-Sound (TRUS)-Magnetic Resonance Imaging (MRI) fusion image of a prostate gland of a subject, comprises the following steps: (a) inputting an MRI scan of the prostate gland of the subject; (b) segmenting the MRI scan to produce at least one segmented MRI contour surface of the organ, the contour comprising a plurality of three-dimensional (3D) landmark points; (c) inputting a TRUS scan of the prostate gland of the subject; (d) segmenting the TRUS scan to produce at least one segmented TRUS contour surface of the prostate gland, the contour comprising a plurality of 3D landmark points, wherein the at least one MRI contour surface and the at least one TRUS contour surface correspond to the same anatomical surface; (e) resampling the TRUS and MRI contours to a common geometric space; (f) computing a linear transformation that maps the MRI contour surface onto the TRUS contour surface, the linear transformation being an affine transformation estimated by minimization of the matching cost between the plurality of landmark points on the MRI contour and the plurality of landmark points on the TRUS contour; (g) applying the linear transformation to the MRI contour points to obtain linearly transformed (LT) MRI contour points; (h) computing a local shape descriptor for each LT landmark point of the MRI contour surface and each landmark point of the TRUS contour surface; (i) computing an optimal assignment between the LT landmark MRI and TRUS contour surface points that minimizes a matching cost criterion between the shape descriptors of the matched points, the optimal assignment defining a sparse vector field mapping MRI contour points onto TRUS contour points; (j) computing a dense deformation field by smooth interpolation of the sparse vector field to map any point of the whole MRI volume onto a point of the TRUS volume; and (k) applying the linear and non-linear mapping of steps (f) through (j) to map points of the MRI image into the TRUS image; wherein the performance of steps (a) through (k) generates a Trans-Rectal Ultra-Sound-Magnetic Resonance Imaging fusion image of the prostate of the subject.

As used throughout, the term “Trans-Rectal Ultra-sound (TRUS)-Magnetic Resonance Imaging (MRI) fusion image” is used interchangeably having all the same meanings and qualities with “Magnetic Resonance Imaging (MRI)-Trans-Rectal Ultra-sound (TRUS), MRI-TRUS, and TRUS-MRI.

As used herein, the terms “Magnetic Resonance Imaging” and “MRI” are used interchangeably having all the same meanings and qualities, refer to a phenomenon in which high frequency energy is incident onto the atomic nucleus magnetized by the magnetic field, and the atomic nucleus in a low energy state is excited by absorbed high frequency energy. As a result, the atomic nucleus then reaches a high energy state. Atomic nuclei have different resonance frequencies according to the types thereof, and resonance is affected by the intensity of the magnetic field. The human body includes multitudinous atomic nuclei, such as ¹H, ²³Na, ³¹P, ¹³C, etc., which exhibit a magnetic resonance phenomenon. In general, a proton is used to generate a magnetic resonance image. MRI may also be termed “nuclear magnetic resonance imaging (NMRI)” or “magnetic resonance tomography (MRT)”.

In response to a radio frequency (RF) pulse having high frequency energy is temporarily applied to a subject, a magnetic resonance signal is emitted from the subject. The magnetic resonance signal emitted from the subject may be classified according to a type of the RF pulse. Thus, a response to a general RF pulse is referred to as a free induction decay (FID), and a response to a refocusing RF pulse is referred to as an echo signal.

In clinical practice, MRI is used to distinguish between tissues (e.g. pathologic tissue such as a tumor from normal tissue) exploiting the different magnetic properties of tissue: decay times (transverse relaxation time, T₂, caused by the intrinsic spin-spin interaction; longitudinal relaxation, T₁, the spin-lattice relaxation time), and proton density. From these, physiological tissue parameters such as diffusion, perfusion, etc. can be derived.

Single MRI images are called slices. The images can be stored on a computer or printed on film. In one embodiment, MRI is performed prior to an operative or invasive medical technique. In another embodiment, MRI is performed concurrent with an operative or invasive medical technique. In one embodiment, MRI prostate imaging is performed by any MRI technique known in the art, for example T2-weighted, diffusion weighted, and Dynamic contrast enhanced.

As used herein, the terms “Trans-Rectal Ultra-Sound” or “TRUS”, refers to an ultrasound technique that is used to view a man's prostate and surrounding tissues. The ultrasound transducer (probe) is inserted into the rectum and sends sound waves through the wall of the rectum into the prostate gland, which is located directly in front of the rectum. TRUS may also be called prostate sonogram or endorectal ultrasound. In one embodiment, TRUS is performed concurrent with an operative or invasive medical technique. Modern TRUS transducers enable the automatic generation of a set of thin contiguous image slices that sample the prostate anatomy from its base to its apex.

In one embodiment this invention comprises, a method for generating a Computerized Tomography (CT)-Magnetic Resonance Imaging (MRI) fusion image of a prostate gland of a subject, comprises the following steps: (a) inputting an MRI scan of the prostate gland of the subject; (b) segmenting the MRI scan to produce at least one segmented MRI contour surface of the organ, the contour comprising a plurality of three-dimensional (3D) landmark points; (c) inputting a CT scan of the prostate gland of the subject; (d) segmenting the CT scan to produce at least one segmented CT contour surface of the prostate gland, the contour comprising a plurality of 3D landmark points, wherein the at least one MRI contour surface and the at least one CT contour surface correspond to the same anatomical surface; (e) resampling the CT and MRI contours to a common geometric space; (f) computing a linear transformation that maps the MRI contour surface onto the CT contour surface, the linear transformation being an affine transformation estimated by minimization of the matching cost between the plurality of landmark points on the MRI contour and the plurality of landmark points on the CT contour; (g) applying the linear transformation to the MRI contour points to obtain linearly transformed (LT) MRI contour points; (h) computing a local shape descriptor for each LT landmark point of the MRI contour surface and each landmark point of the CT contour surface; (i) computing an optimal assignment between the LT landmark MRI and CT contour surface points that minimizes a matching cost criterion between the shape descriptors of the matched points, the optimal assignment defining a sparse vector field mapping MRI contour points onto CT contour points; (j) computing a dense deformation field by smooth interpolation of the sparse vector field to map any point of the whole MRI volume onto a point of the CT volume; and (k) applying the linear and non-linear mapping of steps (f) through (j) to map points of the MRI image into the CT image; wherein the performance of steps (a) through (k) generates a Computerized Tomography-Magnetic Resonance Imaging fusion image of the prostate of the subject.

As used throughout, the term “Computerized Tomography (CT)-Magnetic Resonance Imaging (MRI) fusion image” is used interchangeably having all the same meanings and qualities with “Magnetic Resonance Imaging (MRI)-Computerized Tomography (CT), MRI-CT, and CT-MRI.

Computerized Tomography is a technology that uses computer-processed x-rays to produce tomographic images (virtual ‘slices’) of specific areas of the scanned object, allowing the user to see what is inside it without cutting it open. As used herein, the terms “Computerized Tomography” or “CT”, may be used interchangeably with all the same qualities and meanings, and refer in one embodiment to a combined series of X-ray images taken from many different angles and computer processed to create cross-sectional images of the bones and soft tissues within a subjects body. A CT scan is a set of one or more contiguous CT images slices of a body part. The set of contiguous slices provides a 3D representation of said body part. In one embodiment, the image is of a subject's prostate gland. In another embodiment, the image is of a portion of a subject's prostate gland. In yet another embodiment, the image is of a prostate gland, or a portion thereof, and adjacent tissue or organs.

In some embodiments of this invention, methods are described for generating fusion images. Similar steps are used in generating a TRUS-MRI fusion image or a CT-MRI fusion image wherein the input data and steps, for example, for segmenting, resampling, computing and applying, and any combination thereof, are performed with a TRUS scan or a CT scan, respectively.

As used herein, the term “contour surface” refers in one embodiment to a delimiting or bounding surface for a three-dimensional (3D) object. In another embodiment, the term “contour surface” refers to a delimiting or bounding curve (or curves) for a two-dimensional (2D) planar image, for example a “slice” of a 3D object. As used herein, the terms “contour surface” and “contour” may be used interchangeably having all the same qualities and meanings.

As used herein, the terms “three-dimensional” and “3D” may be used interchangeably having all the same qualities and meanings. As used herein, the terms “two-dimensional” and “2D” may be used interchangeably having all the same qualities and meanings.

In one embodiment, a contour may be obtained manually. In another embodiment, a contour may be obtained by automatic segmentation. In yet, another embodiment, a contour may be obtained by automatic segmentation and subsequently edited manually. In yet another embodiment, a contour may be obtained

As used herein, the terms “contour point”, “point”, “landmark point”, three-dimensional landmark point” and “landmark” may be used interchangeably having all the same meanings and qualities. In one embodiment, landmarks may be selected pre-operatively, for instance from MRI scanned images. In another embodiment, landmarks may be selected interactively using a graphical interface, for instance from an intraoperative TRUS scan. In another embodiment, landmarks may be selected interactively using a graphical interface, for instance from a CT scan used in external beam radiotherapy for image guided navigation and dose planning.

In one embodiment, a landmark comprises the contour of the prostate. In another embodiment, a landmark comprises the contour of the urethra. In yet another embodiment, a landmark comprises calcifications when observable. Calcifications may be periprostatic or prostatic (including peri-urethral), or any combination thereof. In still another embodiment, a landmark comprises the interface between the central and peripheral zones of the prostate. In a further embodiment, a landmark may be selected from the group comprising the contour of the prostate, the contour of the urethra, calcifications, or an interface between the central and peripheral zones of the prostate, or any combination thereof. Calcifications may be periprostatic or prostatic (including peri-urethral), or any combination thereof. In certain embodiments, in order to increase the local accuracy of an MRI-TRUS fusion or an MRI-CT fusion, it may be useful to complement the contour landmarks with landmarks located in the depth of the prostate thereby providing more information about the deformation field inside the prostate. Each of said landmarks must be visible in both imaging modalities (MRI and TRUS or MRI and CT) to enable their usage in the fusion process

As used herein, the term “segmenting” refers in one embodiment to labelling all the pixels or voxels belonging to the same surface contour using an automatic or semi-automatic (interactive) algorithm. The label enables to distinguish the pixels or voxels of the considered surface contour from all the other pixels or voxels in the images thereby enabling their use in the fusion process described above. In some embodiments, the segmentation algorithm relies on statistical models of the prostate shape and appearance (intensity signal) previously obtained using a large set of manually segmented prostate scans. In one embodiment, segmentation is provided by searching for the optimal model parameters that generate a synthetic 3D prostate that is as similar as possible to the prostate in the scan that is to be segmented. Since the synthetic prostate is already segmented by definition, its projection onto the scan to be segmented provides the desired segmentation. The model based approach can be used for MRI, TRUS, and CT prostate segmentation.

As used herein, the term “resampling” refers in one embodiment to a subsequent conversion from a first digital image to a second digital image. When an image is resampled, the image may also be rescaled in size by reduction or enlargement. For example, a user of a computer aided diagnosis (CAD) system who desires to focus in on a particular feature of a prostate gland, for example a cancerous mass, may use a zoom-in operation to enlarge the selected feature. During the zooming operation, the image is resampled to provide an enlarged view of the area of interest.

As used herein, the term “common geometric space” refers in one embodiment, to bring the scans to the same spatial resolution that is, the same size in physical units, for their voxels.

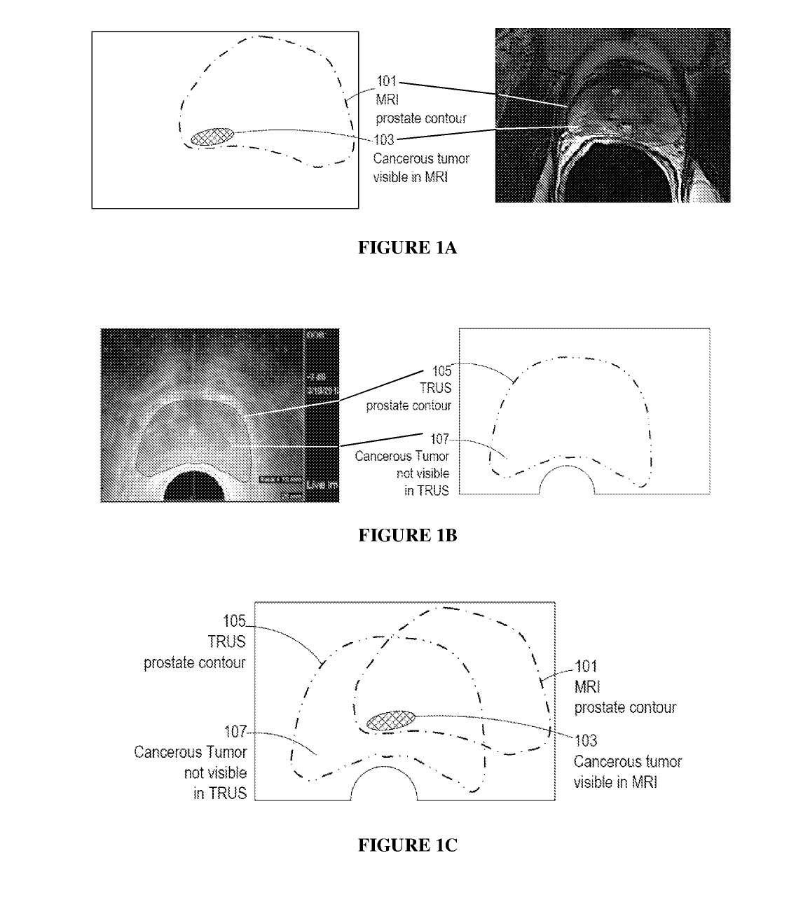

FIGS. 1-2 and 5 provide representative scans and tracings in 2D, illustrating certain steps in generating an MRI-TRUS fusion image. Similar representative illustrations substituting a CT prostate contour scan for the TRUS prostate would depict alternate embodiment of this method, wherein an MRI-CT fusion image would be generated. In addition, note that the depiction of illustrations as 2D in FIGS. 1-2 and 5 is for clarity and simplicity. In certain embodiments, the histogram of the spatial distribution of contour points surrounding a given contour point is three-dimensional. In some embodiments, a fusion image generated is 3D.

According to certain embodiments of the present invention, an MRI contour, for example 101 of FIG. 1A may be manually traced from an MRI image. In other embodiments, contour 101 may be automatically generated. According to still other embodiments, contour 101 is automatically generated and subsequently edited manually. According to a related embodiment, Active Appearance Model (AAM) techniques can be applied to obtain contour 101. According to another related embodiment, active contour techniques can be applied to obtain contour 101.

High resolution MRI scans offer better contrast between the prostate and surrounding tissues than TRUS does. Through a combination of complementary multiparametric MRI sequences (T1w, T2w, DWI, DCE,), it is possible to delineate suspected tumor zones as required for the planning of a focal therapy. In order to benefit from MRI's contrast superiority, certain embodiments of the invention provide for performing the pre-operative planning on MRI scans prior to fusing with intra-operative TRUS. This approach has the advantage of minimizing the portion of the planning made while the patient is already under anesthesia and, eventually, minimizing the overall duration of the procedure.

According to certain embodiments of the present invention, a TRUS contour, for example as shown in FIG. 1B 105, may be manually traced from a TRUS slice. In other embodiments, a contour may be automatically generated. In another embodiment, a contour may be automatically generated and edited manually. According to a related embodiment, an MRI contour, for example as is shown in FIG. 1A 101, may be projected onto the MRI scan and interactively modified manually to obtain TRUS contour, such as is shown in FIG. 1B 105. Alternatively, the projected MRI contour may be used as an initial approximation of the desired TRUS contour that can be obtained by applying a curve evolution algorithm to the initial approximation, e.g. level sets, in order to make it converge into the desired TRUS contour.

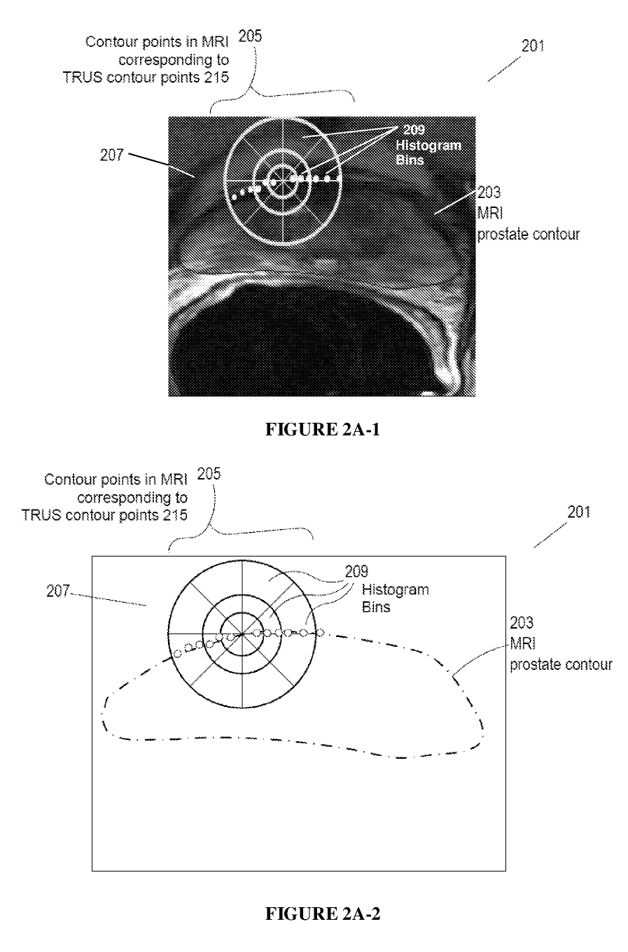

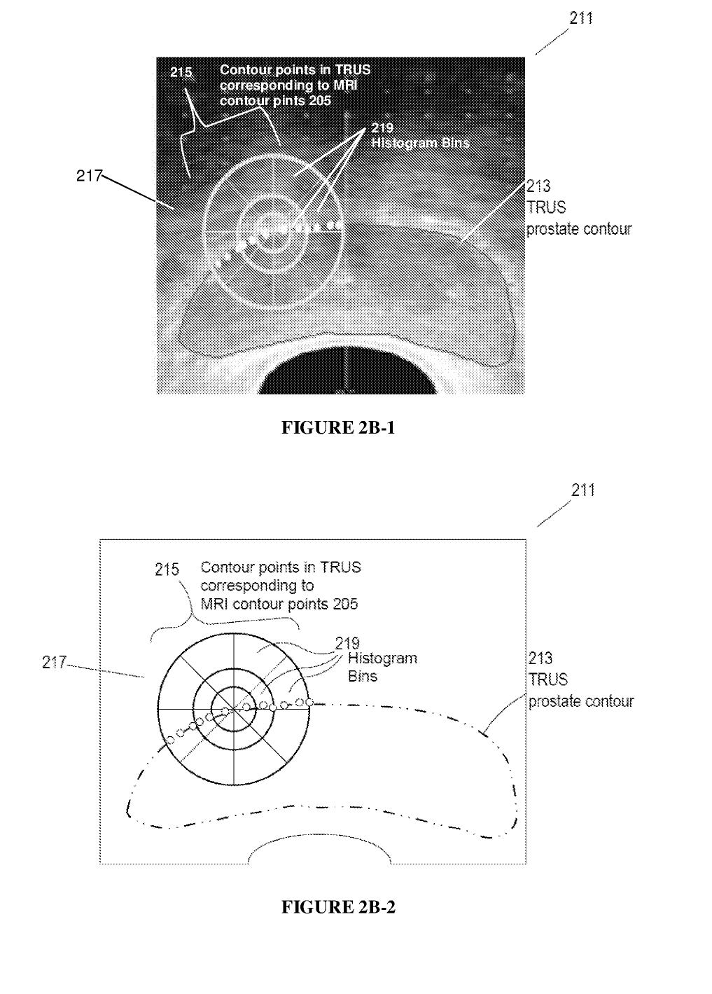

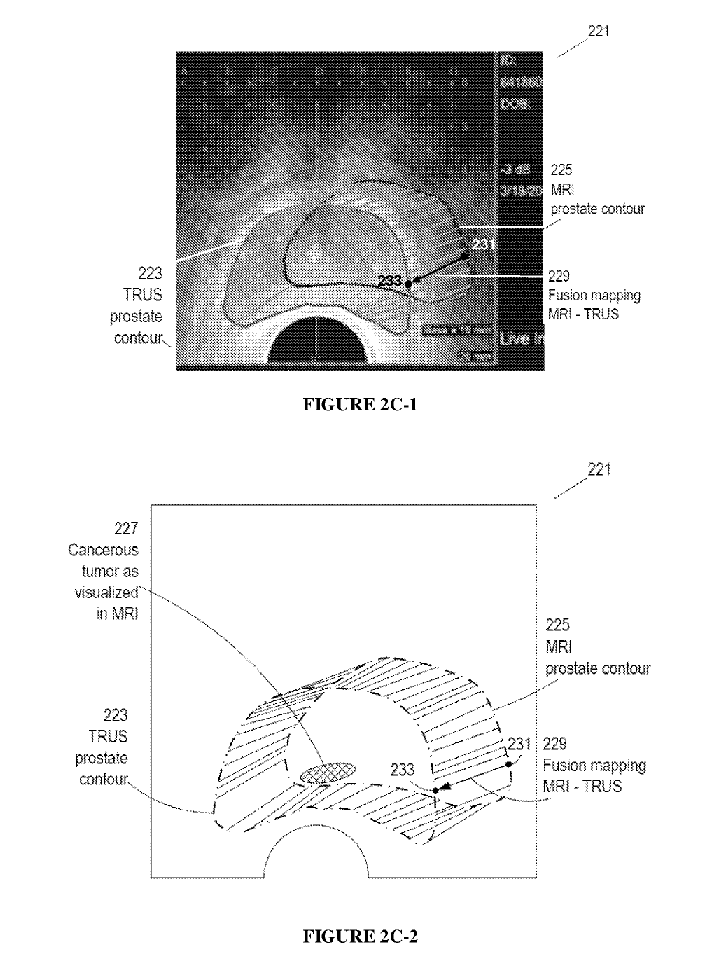

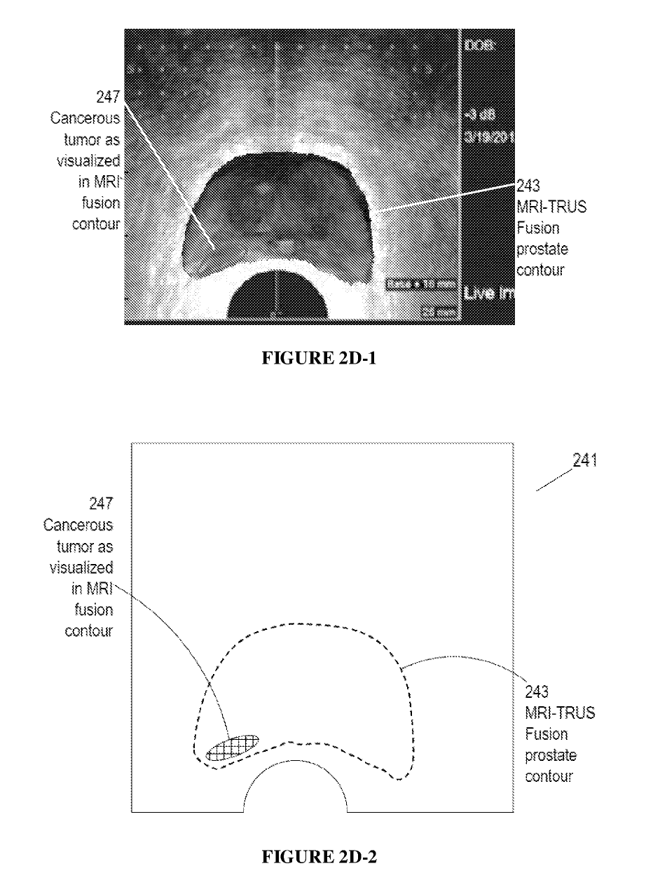

In one embodiment, a shape context for a landmark point provides a rich description of the spatial distribution of neighboring points. In one embodiment, this rich description of spatial distribution of neighboring points is termed a “local shape descriptor”. For example, for a given MRI contour point, as shown in FIGS. 2A-1 and 2A-2 205, the spatial distribution of neighbor points is encoded by the normalized counts of a log distance-polar histogram 207 having histogram bins 209. Histogram bins 209 may be positioned at constant angular spacing, but logarithmically spaced in the radial direction. The shape context is inherently shift invariant and can be easily made scale and rotation invariant by appropriate normalization. Similarly, for TRUS and CT contour points, the spatial distribution of neighbor points is encoded by the normalized counts of a log distance-polar histogram 207 having histogram bins 209. Histogram bins 209 may be positioned at constant angular spacing, but logarithmically spaced in the radial direction. The shape context is inherently shift invariant and can be easily made scale and rotation invariant by appropriate normalization.

Once the descriptor is assigned to each landmark point in both modalities, MRI and TRUS, a cost is computed for the matching of each landmark pairs across the modalities. The cost, Ci,j, of matching between landmark points i and j belonging to MRI and TRUS images, respectively, is computed as shown by Equation 1:

$$\begin{matrix}Equation 1 & \\ {C}_{i , j} = \frac{1}{2} {\sum }_{k = 1}^{K} \frac{{[ g ( k ) - h ( k ) ]}^{2}}{g ( k ) + h ( k )} & ( 1 )\end{matrix}$$

Where g(k) and h(k) stand for the counts in bin k of the normalized shape context histogram at points i (MRI image) and j (TRUS image), respectively. The total number of histogram bins is K. Optimal matches are computed using a shortest augmenting path algorithm well known in prior art.

The matched landmark points define a sparse deformation field from which a smooth and dense deformation field is computed. For this purpose a 3D b-spline grid is fitted that transforms the MRI landmark points into the matched TRUS points with a minimum error and smoothly interpolate the deformation field anywhere else. The resulting dense deformation field is consecutively used to map the MRI voxels located inside the prostate contour into corresponding TRUS voxels, thereby generating the fusion image, and including artifacts visualized by MRI within the MRI contour, such as cancerous tumor as shown at 227 (2C-2).

In one embodiment, for a TRUS contour point, for example as shown in FIGS. 2B-1 and 2B-2, 215, the spatial distribution of neighbor points may be encoded by the normalized counts of a log distance-polar histogram 217 having histogram bins 219. The histogram 217 may be computed exactly in the same fashion described above for an MRI contour point 205.

As used herein, the term “subject” refers in one embodiment to a mammalian subject. In one embodiment, a subject is a human subject.

In one embodiment, MRI, TRUS, CT, MRI-TRUS, MRI-CT images may include the entire prostate gland. In another embodiment, MRI, TRUS, CT, MRI-TRUS and MRI-CT images may include less than the entire prostate gland. In yet another embodiment, MRI, TRUS, CT MRI-TRUS and MRI-CT images may include only a part of a prostate gland.

Embodiments of the present invention provide an approach to focal therapies, which may be widely disseminated, for example, in all existing brachytherapy systems and require only an updated version of the treatment planning system (TPS). According to certain embodiments of the invention, selective interstitial seed implant performed with TRUS guidance and planned intra-operatively using fused images of multi-parametric MRI and histology maps imported into the TPS is the most efficient strategy, and is implemented via methods disclosed herein. The incorporation and use of fused MRI-TRUS images, for example pre-operative MRI images and intra-operative TRUS images, during medical procedures which may include surgery, biopsy and seed implantation, may benefit prostate cancer subjects undergoing local therapy treatment(s). Similarly, incorporation and use of fused MRI-CT images, for example pre-operative MRI images and CT images, during medical procedures which may include external beam radiotherapy, surgery, biopsy and seed implantation, may benefit prostate cancer treatment accuracy.

In one embodiment, a focal therapy may be a diagnostic procedure. In another embodiment, a focal therapy may be an intervention procedure. In yet another embodiment, a focal therapy may be a therapeutic procedure. In still another embodiment, a focal therapy may be a diagnostic procedure, an intervention procedure, a therapeutic procedure, or any combination thereof. Examples of focal therapies include surgical procedures known in the art, biopsy, an image guided biopsy, seed implantation, prostatectomy, robotic prostatectomy, brachytherapy, cryotherapy, high intensity focalized ultrasound therapy, vascular targeted photodynamic therapy, or surgery for removal of a tumor, or any combination thereof.

Additionally, the incorporation and use of fused MRI-TRUS images, for example pre-operative MRI images and intra-operative TRUS images, during medical procedures, for example biopsy, may benefit subjects suspected of having prostate cancer and undergoing a local biopsy regime, as the fused image may provide improved accuracy for determining the position of a target. In an alternate embodiment, the incorporation and use of fused MRI-CT images, for example pre-operative MRI images and CT images, during medical procedures, for example biopsy, may benefit subjects suspected of having prostate cancer and undergoing a local biopsy regime, as the fused image may provide improved accuracy for determining the position of a target. In one embodiment, a target may be the entire prostate gland. In another embodiment, a target may be a portion of the prostate gland. In yet another embodiment, a target may be a lesion within a prostate gland, for example a cancerous or non-cancerous tumor, or pre-hyperplasic tissue.

Erection problems are one of the serious side effects of radical prostatectomy. The nerves that control a man's ability to have an erection lie next to the prostate gland. They often are damaged or removed during surgery.

Improved accuracy during a medical procedure may in some embodiments, provide details for determining positions to avoid during surgical procedures. Fused images generated by the methods of this invention and used to improve target therapy, can also be used to avoid damaging tissue in the surrounding area of the prostate gland. In one embodiment, methods of this invention generating fused images provide a visualization and localization of the neurovascular bundle adjacent to the prostate gland. In one embodiment, methods of use of this invention improving the accuracy of determining the location to target during said medical procedure and further comprise improving the accuracy of determining a location to avoid during the medical procedure in order that the neurovascular bundle is not damaged. In one embodiment, a method of use of this invention avoids damaging the neurovascular bundle during robotic prostatectomy.

Various embodiments of the invention provide for smooth interpolation to extend warping to the whole volume inside the prostate contour, thereby enabling volume fusion between MRI and TRUS prostate images, or MRI and CT prostate images.

In one embodiment, this invention provides a method of using a fused Trans-Rectal Ultra-Sound (TRUS)-Magnetic Resonance Imaging (MRI) image of a prostate of a subject for improving the accuracy of determining a location of target for a medical procedure, the method comprising the following steps: (a) inputting an MRI scan of the prostate gland of the subject; (b) segmenting the MRI scan to produce at least one segmented MRI contour surface of the organ, the contour comprising a plurality of three-dimensional (3D) landmark points; (c) inputting a TRUS scan of the prostate gland of the subject; (d) segmenting the TRUS scan to produce at least one segmented TRUS contour surface of the prostate gland, the contour comprising a plurality of 3D landmark points, wherein the at least one MRI contour surface and the at least one TRUS contour surface correspond to the same anatomical surface; (e) resampling the TRUS and MRI contours to a common geometric space; (f) computing a linear transformation that maps the MRI contour surface onto the TRUS contour surface, the linear transformation being an affine transformation estimated by minimization of the matching cost between the plurality of landmark points on the MRI contour and the plurality of landmark points on the TRUS contour; (g) applying the linear transformation to the MRI contour points to obtain linearly transformed (LT) MRI contour points; (h) computing a local shape descriptor for each LT landmark point of the MRI contour surface and each landmark point of the TRUS contour surface; (i) computing an optimal assignment between the LT landmark MRI and TRUS contour surface points that minimizes a matching cost criterion between the shape descriptors of the matched points, the optimal assignment defining a sparse vector field mapping MRI contour points onto TRUS contour points; (j) computing a dense deformation field by smooth interpolation of the sparse vector field to map any point of the whole MRI volume onto a point of the TRUS volume; and (k) applying the linear and non-linear mapping of steps (f) through (j) to map points of the MRI image into the TRUS image; wherein the performance of steps (a) through (k) generates a Trans-Rectal Ultra-Sound-Magnetic Resonance Imaging fusion image of the prostate, and wherein the fused image provides improved accuracy of determining the location of target for the medical procedure.

In an alternate embodiment, a method of this invention provides using a fused Computerized Tomography (CT) image-Magnetic Resonance Imaging (MRI) image of a prostate of a subject for improving the accuracy of determining a location of target for a medical procedure, the method comprising the steps of (a) through (k) above.

In one embodiment, this invention provides a method for treating or diagnosing a subject having prostate cancer, or suspected of having cancer using a fused Trans-Rectal Ultra-Sound (TRUS)-Magnetic Resonance Imaging (MRI) image of a prostate gland of the subject, the method of treatment or diagnosis comprising a surgical procedure; wherein at the time the surgical procedure the method includes the steps of (a) through (k) above.

In an alternate embodiment, this invention provides a method of this invention for treating or diagnosing a subject having prostate cancer, or suspected of having cancer using a fused Computerized Tomography (CT)-Magnetic Resonance Imaging (MRI) image of a prostate gland of the subject, the method of treatment or diagnosis comprising an external beam radiotherapy procedure; wherein at the time the procedure the method includes the steps of (a) through (k) above.

In one embodiment, a subject has prostate cancer. In another embodiment, a subject is suspected of having prostate cancer.

In one embodiment, the prostate cancer is an early stage prostate cancer. In another embodiment, the prostate cancer is late stage prostate cancer. Late stage prostate cancer includes prostate cancer that is advanced. In yet another embodiment, the prostate cancer is castration resistant prostate cancer. In another embodiment, the prostate cancer is metastatic prostate cancer. In one embodiment, a subject has benign prostatic hyperplasia.

As used herein, the term “treating” refers to both therapeutic treatment and prophylactic or preventative measures, wherein the object is to prevent or lessen the targeted pathologic condition or disorder as described herein. Thus, in one embodiment, treating may include directly affecting or curing, suppressing, inhibiting, preventing, reducing the severity of, delaying the onset of, reducing symptoms associated with for example prostate cancer. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

As used herein, the term “diagnosis” refers in one embodiment to the identification of the disease (prostate cancer) at any stage of its development, and also includes the determination of predisposition of a subject to develop the disease. In one embodiment of the invention, diagnosis of prostate cancer occurs prior to the manifestation of symptoms. Subjects with a higher risk of developing the disease are of particular concern. The diagnostic method of the invention also allows confirmation of prostate cancer in a subject suspected of having prostate cancer. “Differential diagnosis” refers to differentiating between tumor and metastasis, thereby facilitating the differentiation between an individual having metastasis-free prostate cancer and an individual having metastatic prostate cancer.

As used herein in the specification and claims, the forms “a,” “an” and “the” include singular as well as plural references unless the context clearly dictates otherwise.

As used herein, the term “comprising” is intended to mean that the method includes the recited steps, but not excluding others which may be optional. By the phrase “consisting essentially of” it is meant a method that includes the recited steps but excludes other steps that may have an essential significant effect on the performance of the method. “Consisting of” shall thus mean excluding steps other than those listed. Embodiments defined by each of these transition terms are within the scope of this invention.

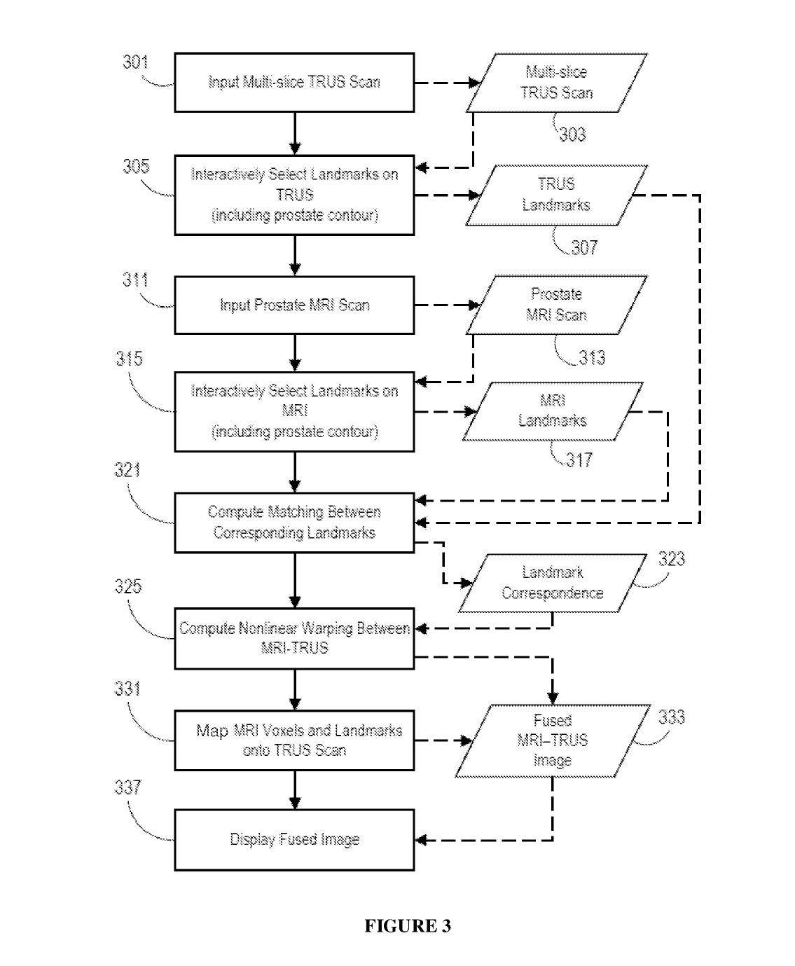

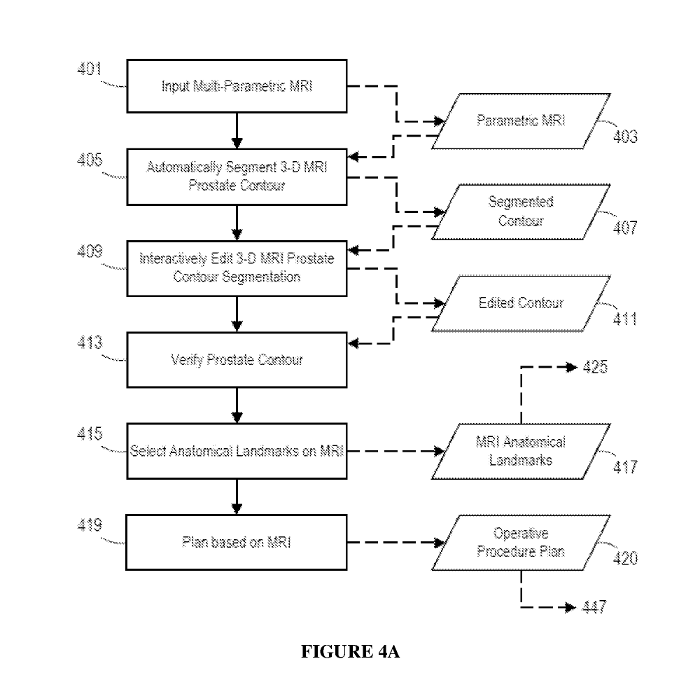

FIG. 3 provides a flowchart of a method for generating a TRUS-MRI image fusion, according to an embodiment of the present invention. In a step 301 a multi-slice TRUS prostate scan 303 is input during the operative procedure (“intraoperatively”), and in a step 305, TRUS landmarks 307 on TRUS scan 303 are interactively selected, including the TRUS prostate contour. In a step 311, a prostate MRI scan 313 is input, and in a step 315 MRI landmarks 317 are interactively selected, including the MRI prostate contour. Then, in a step 321 minimum cost landmark correspondence 323 is computed to match between corresponding landmarks of TRUS landmarks 307 and MRI landmarks 317. Next, in a step 325 a dense non-linear warping between the MRI contour and the TRUS contour is computed using landmark correspondence 323. Applying said warping to map MRI prostate voxels into the TRUS prostate volume to obtain a fused MRI-TRUS image 333, and in a step 337 fused image 333 is displayed. Example 1 provides an example of a fused image for a single TRUS slice (2D TRUS-MRI fused image), wherein to obtain information in 3D, 3D MRI and TRUS scans would be used. A similar flowchart substituting a CT scan for TRUS scan depicts steps of a method for generating a CT-MRI fused image.

According to various embodiments of the present invention, given prostate MRI scan 313 and 3D (multi-slice) TRUS scan 303 of the same patient, the user may interactively select TRUS landmarks 307 and MRI landmarks 317 using the computer mouse on both scans. These may include the contour of the prostate, the urethra, and other landmarks such as calcification, or the interface between the central and peripheral zones. Calcifications may be periprostatic or prostatic (including peri-urethral), or any combination thereof.

To increase the local accuracy of the fusion, various embodiments of the invention complement the contour landmarks with landmarks located in the depth of the prostate, thereby providing more information about the deformation field inside the prostate.

In a related embodiment, points corresponding to the anatomical landmarks selected on the prostate MRI may be marked interactively on the TRUS volume. A linear affine warping may then be computed between the pairs of corresponding MRI-TRUS landmark points and applied to the prostate MRI contour in order to project it onto the TRUS volume.

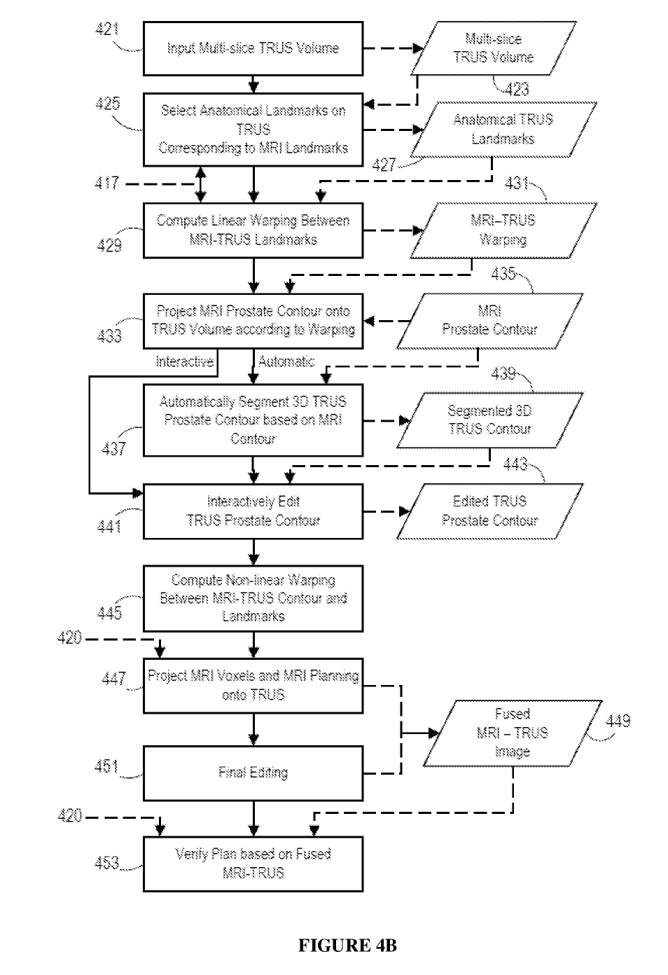

FIG. 4A is a flowchart of a method for establishing MRI image landmarks according to another embodiment of the present invention. In a step 401 a multi-parametric MRI image 403 is input, and in a step 405 multi-parametric MRI image 403 is automatically segmented into a set of segmented contours 407 defining the prostate, the targeted lesion (tumor) and, optionally, other anatomical structures/landmarks such as the urethra, the interface between the central and peripheral zones. Then, in a step 409 segmented contours set 407 is interactively edited to obtain an edited contour 411, which is verified in a step 413. In a step 415, MRI anatomical landmarks 417 may be selected, and in a step 419, operational procedure planning 420 may be performed, based on the MRI.

In the case of brachytherapy planning, the planning includes finding the optimal spatial distribution of the radioactive seeds on the MRI images to obtain the desired therapeutic effect. MRI anatomical landmarks 417 and planning 420 may be made available to a method for TRUS-MRI image fusion, or CT-MRI image fusion (not shown), performed at the time of a medical procedure, which is detailed in FIG. 4B, as discussed below.

The above method steps include the first preoperative step of automatic segmentation of the prostate contours on each MRI slice for further usage during the planning. Even for an expert clinician, accurately drawing the prostate contour on MRI slices is a difficult task due to the large variations of prostate shape between subjects, the lack of a continuous prostate boundary and the similar intensity profiles of the prostate and surrounding tissues. In order to obtain a robust segmentation under these challenging conditions, related embodiments of the invention may utilize Active Appearance Models (AAM), which enforce prior knowledge on prostate shape and MRI appearance. In AAM, a parametric 3D model of the prostate is learned, off-line, from a set of manually-segmented and co-registered MRI prostate scans acquired on different subjects. Once the model is learned, “synthetic” prostate instances can be generated with variable shape and appearance, by modifying the values of the model parameters. The segmentation of the prostate contour in a new scan is an optimization process in which the model's parameters are adjusted to minimize the discrepancy between the synthetic prostate and the voxels sampled in the new scan.

According to these related embodiments, the contour obtained by automatic segmentation is displayed in a user interface (UI) window and is manually edited through a set of control points interactively movable with a computer mouse. The verified contour provides a set of landmark points that is used in the fusion with TRUS. In order to obtain an accurate 3D fusion, other related embodiments provide for complementing the contour points with anatomical landmark points visible both in MRI and TRUS such as the urethra, periprostatic, prostatic (including peri-urethral) calcifications, peripheral zone and transitional zone landmarks, etc. The preoperative planning is thus completed in a way that minimizes the planning tasks that remain to be done during the operation.

FIG. 4B is a flowchart of another method for TRUS-MRI image fusion according to a one embodiment of the present invention. In a step 421 a multi-slice TRUS volume 423 is input, from which anatomical TRUS landmark data 427 may be selected in a step 425, corresponding to MRI landmarks 417 (see FIG. 4A and description above). Anatomical TRUS landmarks 427 and MRI landmarks 417 may then used in a step 429 to compute linear warping 431 between MRI landmarks 417 and TRUS landmarks 427. Then, in a step 433 an MRI prostate contour 435 is projected onto TRUS volume 423 using the computed linear warping 431.

According to one embodiment of the invention, a smooth nonlinear warping is computed between TRUS landmarks 427 and MRI landmarks 417, and the smooth interpolation is used to estimate a dense deformation field that maps all the MRI voxels onto corresponding TRUS voxels. In another related embodiment, “rich shape descriptors” according to the known “shape context” formalism may be used to match between MRI and TRUS landmarks.

According to a first related embodiment, in a step 437 3D TRUS contour 439 is automatically output based on MRI contour 435, and in a following step 441 an edited TRUS prostate contour 443 is output interactively. According to a second related embodiment, step 437 is skipped, and step 433 proceeds directly to step 441, so that edited TRUS contour 443 is based solely on interactive editing.

Next, in a step 445, non-linear warping between the MRI-TRUS contour and the landmarks is computed, and in a step 447, MRI voxels and planning 420 (from step 419 in FIG. 4A), including targeted tumor contour, are projected onto a fused MRI-TRUS image 449. In a step 451, final editing is performed on MRI-TRUS image 449, and then a step 453 verifies plan 420 based on fused image 449. According to a related embodiment of the invention, the set of corresponding MRI-TRUS points, together with the landmarks used for the initial affine warping are used to compute the non-linear warping in step 445, to accurately project the MRI prostate, including the surgical planning, onto the TRUS images, thereby performing the fusion. In another related embodiment, the planning on the fused modalities is modified manually and validated.

Additional embodiments of the invention provide a method to avoid a progressive loss in positioning accuracy in cases where the prostate is displaced and/or deformed during the operative procedure (such as by the insertion of radioactive brachytherapy seeds via needles or progressing tissue resection during robotic prostatectomy). These embodiments compensate for such effects by performing a periodic alignment update between the first 3D TRUS scan (the one used for MRI-TRUS fusion) and updated TRUS scans acquired during the operative procedure. This alignment requires intra-modality registration (TRUS-TRUS) with a nonlinear warping, because the effect of deformation is mostly local. In a related embodiment, an elastic intensity based registration scheme is used for compensation using elastic registration methods known in prior art.

The following example is presented in order to more fully illustrate the preferred embodiments of the invention. It should in no way, however, be construed as limiting the broad scope of the invention.

### Example

### Generating a TRUS-MRI Fused Image

An interactive framework was developed for accurate and robust fusion between prostate preoperative Magnetic Resonance Imaging (MRI) and operative Trans-Rectal Ultra-Sound (TRUS).

An MRI contour of a prostate gland (FIG. 1A, 101) with a cancerous tumor visualized with MRI technology (FIG. 1A, 103) was manually drawn based on the MRI image obtained from a scan of a patient. The area of 103 represents the target area for focal therapy.

Following MRI imaging, a TRUS contour of the prostate gland of FIG. 1A was manually drawn based on an intraoperative scan (FIG. 1B, 105). The cancerous tumor observed by MRI (FIG. 1A, 103) in the region 107 of FIG. 1B was not visible in the TRUS image.

FIG. 1C illustrates the superposition of the MRI contour 101 showing a cancerous tumor 103 of FIG. 1A and the TRUS contour 105 of FIG. 1B.

The initial misalignment between the MRI contour 101 and the TRUS contour 105 showed a significant transverse offset combined with local differences in shape. Consequently, MRI and TRUS contour points needed to be matched. As described below, a descriptor was computed for each landmark in both MRI and TRUS modalities. To ensure robust matching, the descriptor represented the landmarks in a unique yet similar fashion in both modalities.

FIGS. 2A-1 and 2A-2, and 2B-1 and 2B-2 provide representative scans/illustrations of the concept of shape context in two dimensions (2D) of two corresponding contour points, used in the generation of a fused MRI-TRUS image. FIGS. 2A-1 and 2A-2, 205 illustrates contour points in an MRI prostatic image (201) corresponding to TRUS contour points 215, with an MRI contour 203 corresponding to MRI contour 101 of FIG. 1A, For a given MRI contour point 205, the spatial distribution of neighbor points was encoded by the normalized counts of a log distance-polar histogram 207 having histogram bins 209. Histogram bins 209 were positioned at constant angular spacing, but logarithmically spaced in the radial direction. The shape context is inherently shift invariant and may be easily made scale and rotation invariant by appropriate normalization.

FIGS. 2B-1 and 2B-2, 215 illustrate contour points in a TRUS prostatic image (211) corresponding to MRI contour point 205, with a TRUS contour 213 corresponding to TRUS contour 105 of FIG. 1B. For a given TRUS contour point 215, the spatial distribution of neighbor points was encoded by the normalized counts of a log distance-polar histogram 217 having histogram bins 219. The histogram 217 was computed exactly in the same fashion described above for an MRI contour point 205.

Next, the MRI prostate contour was overlaid with the TRUS prostate contour (representatively shown in FIGS. 2C-1 and 2C-2 for a single contour). FIGS. 2C-1 and 2C-2 illustrate the mapping 221 of MRI contour (225), corresponding to MRI contour 101 of FIG. 1A onto TRUS contour (223), corresponding to TRUS contour 105 of FIG. 1B.

For example, an MRI-TRUS mapping 229 transformed a point 231 on MRI contour 225 to a point 233 on TRUS contour 223. Once the descriptor was assigned to each landmark point in both modalities, a cost was computed for the matching of each landmark pairs across the modalities. The cost, of matching between landmark points i and j belonging to MRI and TRUS images, respectively, was computed as shown by Equation 1:

$${C}_{i , j} = \frac{1}{2} {\sum }_{k = 1}^{K} \frac{{[ g ( k ) - h ( k ) ]}^{2}}{g ( k ) + h ( k )} \qquad Equation 1$$

Where g(k) and h(k) stand for the counts in bin k of the normalized shape context histogram at points i (MRI image) and j (TRUS image), respectively. The total number of histogram bins was K. Optimal matches were computed using a shortest augmenting path algorithm.

The matched landmark points defined a sparse deformation field from which a smooth and dense deformation field, was computed. For this purpose a 3D b-spline grid was fitted that transforms the MRI landmark points into the matched TRUS points with a minimum error and smoothly interpolate the deformation field anywhere else. The resulting dense deformation field was consecutively used to map the MRI voxels located inside the prostate contour into corresponding TRUS voxels, thereby generating the fusion image, and including artifacts visualized by MRI within the MRI contour, such as the cancerous tumor 227 (FIG. 2C-2).

FIGS. 2D-1 and 2D-2 show a representative 2D fused TRUS-MRI prostate image 241, and tracing thereof. Image 241 features MRI prostate voxels mapped inside a TRUS image and delimited by a TRUS contour 223. Cancerous tumor 227 as visualized in MRI was also mapped to a contour 247 in its correct location relative to contour 223. As a next step, for instance to treat a cancer patient, the region of the prostate corresponding to the target area within contour 247 would be treated, such as with focal brachytherapy or focal robotic prostatectomy.

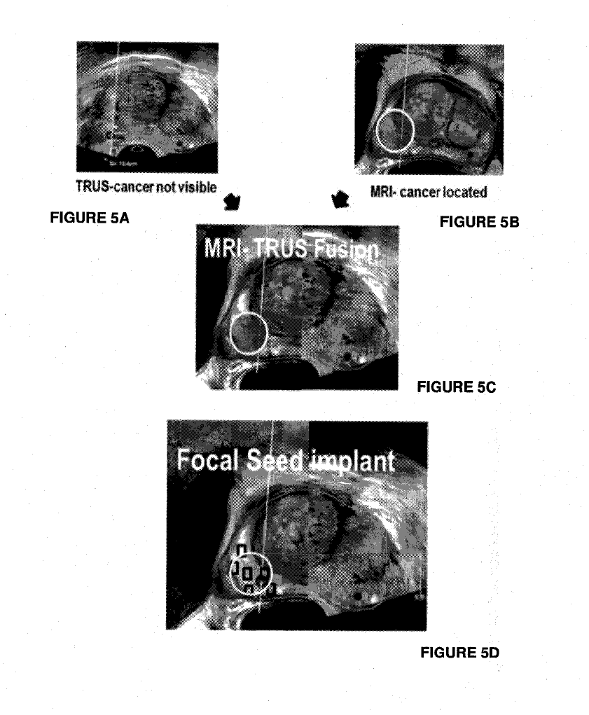

FIGS. 5A-D present embodiments of TRUS and MRI scans (5A and 5B, respectively) and a fused MRI-TRUS image (5C), visually showing the images from the initial input scans through to the generated fused image, and use of the fused image for targeting a cancer (5D) during a medical procedure, wherein the focal seed implant was targeted to a well-defined area (boxes). Each box represents a location for a seed implant during brachytherapy.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for generating a Trans-Rectal Ultra-Sound (TRUS)-Magnetic Resonance Imaging (MRI) fusion image of a prostate gland of a subject, said method comprising the following steps:
(a) inputting an MRI scan of the prostate gland of said subject;
(b) segmenting the MRI scan to produce at least one segmented MRI contour surface of the organ, said contour comprising a plurality of three-dimensional (3D) landmark points;
(c) inputting a TRUS scan of the prostate gland of said subject;
(d) segmenting the TRUS scan to produce at least one segmented TRUS contour surface of the prostate gland, said contour comprising a plurality of 3D landmark points, wherein the at least one MRI contour surface and the at least one TRUS contour surface correspond to the same anatomical surface;
(e) resampling the TRUS and MRI contours to a common geometric space;
(f) computing a linear transformation that maps the MRI contour surface onto the TRUS contour surface, the linear transformation being an affine transformation estimated by minimization of the matching cost between the plurality of landmark points on the MRI contour and the plurality of landmark points on the TRUS contour;
(g) applying said linear transformation to the MRI contour points to obtain linearly transformed (LT) MRI contour points;
(h) computing a local shape descriptor for each LT landmark point of the MRI contour surface and each landmark point of the TRUS contour surface;
(i) computing an optimal assignment between said LT landmark MRI and TRUS contour surface points that minimizes a matching cost criterion between the shape descriptors of the matched points, said optimal assignment defining a sparse vector field mapping MRI contour points onto TRUS contour points;
(j) computing a dense deformation field by smooth interpolation of said sparse vector field to map any point of the whole MRI volume onto a point of the TRUS volume;
and
(k) applying the linear and non-linear mapping of steps (f) through (j) to map points of the MRI image into the TRUS image;
wherein the performance of steps (a) through (k) generates a Trans-Rectal Ultra-Sound-Magnetic Resonance Imaging fusion image of the prostate of said subject.

2. The method of claim 1, wherein said at least one contour surface comprises an external surface of the prostrate or a portion thereof, a contour of an internal surface of the prostate or a portion thereof, a contour of a transitional zone of the prostate or a portion thereof, a contour of a central zone of the prostate or a portion thereof, a contour of a peripheral zone of the prostate or a portion thereof, a contour of an interface between a central zone and a peripheral zone of the prostate or a portion thereof, a contour of a surface bordering the prostate and the urethra or a portion thereof, a contour based on observable calcifications, or any combinations thereof, or any combination thereof.

3. (canceled)

4. The method of claim 1, wherein said minimizes a matching cost criterion of step (i) is computed according to the count distribution of contour points falling within a plurality of histogram bins neighboring each landmark point.

5. The method of claim 1, wherein the dense deformation field is constrained to be smooth and invertible.

6. (canceled)

7. The method of claim 1, wherein said subject is undergoing a focal procedure, wherein said focal procedure comprises a prostatectomy, a robotic prostatectomy, a biopsy, an image guided biopsy, brachytherapy, cryotherapy, a high intensity focalized ultrasound therapy, a vascular targeted photodynamic therapy, a radiotherapy, an external beam radiotherapy, or a surgery for removal of a tumor, or any combination thereof.

8. (canceled)

9. (canceled)

10. The method of claim 1, wherein said method further visualizes and locates the neurovascular bundle adjacent to said prostate gland.

11. A method of using a fused Trans-Rectal Ultra-Sound (TRUS)-Magnetic Resonance Imaging (MRI) image of a prostate of a subject for improving the accuracy of determining a location of target for a medical procedure, said method comprising the following steps:
(a) inputting an MRI scan of the prostate gland of said subject;
(b) segmenting the MRI scan to produce at least one segmented MRI contour surface of the organ, said contour comprising a plurality of three-dimensional (3D) landmark points;
(c) inputting a TRUS scan of the prostate gland of said subject;
(d) segmenting the TRUS scan to produce at least one segmented TRUS contour surface of the prostate gland, said contour comprising a plurality of 3D landmark points, wherein the at least one MRI contour surface and the at least one TRUS contour surface correspond to the same anatomical surface;
(e) resampling the TRUS and MRI contours to a common geometric space;
(f) computing a linear transformation that maps the MRI contour surface onto the TRUS contour surface, the linear transformation being an affine transformation estimated by minimization of the matching cost between the plurality of landmark points on the MRI contour and the plurality of landmark points on the TRUS contour;
(g) applying said linear transformation to the MRI contour points to obtain linearly transformed (LT) MRI contour points;
(h) computing a local shape descriptor for each LT landmark point of the MRI contour surface and each landmark point of the TRUS contour surface;
(i) computing an optimal assignment between said LT landmark MRI and TRUS contour surface points that minimizes a matching cost criterion between the shape descriptors of the matched points, said optimal assignment defining a sparse vector field mapping MRI contour points onto TRUS contour points;
(j) computing a dense deformation field by smooth interpolation of said sparse vector field to map any point of the whole MRI volume onto a point of the TRUS volume;
and
(k) applying the linear and non-linear mapping of steps (f) through (j) to map points of the MRI image into the TRUS image;
wherein the performance of steps (a) through (k) generates a Trans-Rectal Ultra-Sound-Magnetic Resonance Imaging fusion image of said prostate, and wherein said fused image provides improved accuracy of determining said location of target for said medical procedure.

12. The method of claim 11, wherein said at least one contour surface comprises an external surface of the prostrate or a portion thereof, a contour of an internal surface of the prostate or a portion thereof, a contour of a transitional zone of the prostate or a portion thereof, a contour of a central zone of the prostate or a portion thereof, a contour of a peripheral zone of the prostate or a portion thereof, a contour of an interface between a central zone and a peripheral zone of the prostate or a portion thereof, a contour of a surface bordering the prostate and the urethra or a portion thereof, a contour based on observable calcifications, or any combinations thereof, or any combination thereof.

13. (canceled)

14. The method of claim 11, wherein said minimizes a matching cost criterion of step (i) is computed according to the count distribution of contour points falling within a plurality of histogram bins neighboring each landmark point.

15. The method of claim 11, wherein the dense deformation field is constrained to be smooth and invertible.

16. (canceled)

17. The method of claim 11, wherein said medical procedure comprises a focal procedure, wherein said focal procedure comprises a prostatectomy, a robotic prostatectomy, a biopsy, an image guided biopsy, brachytherapy, cryotherapy, a high intensity focalized ultrasound therapy, a vascular targeted photodynamic therapy, a radiotherapy, an external beam radiotherapy, or a surgery for removal of a tumor, or any combination thereof.

18. (canceled)

19. (canceled)

20. The method of claim 11, wherein said target is the complete prostate gland, a region of the prostate gland, a tumor within the prostate gland, or any combination thereof.

21. The method of claim 11, wherein said subject has prostate cancer or is suspected of having prostate cancer.

22. The method of claim 11, wherein said fused image provides a visualization and localization of the neurovascular bundle adjacent to the prostate gland, wherein said improving the accuracy of determining the location to target during said medical procedure further comprises improving the accuracy of determining a location to avoid during said medical procedure in order that said neurovascular bundle is not damaged.

23. (canceled)

24. A method of treating or diagnosing a subject having prostate cancer, or suspected of having cancer using a fused Trans-Rectal Ultra-Sound (TRUS)-Magnetic Resonance Imaging (MRI) image of a prostate gland of said subject, said method of treatment or diagnosis comprising a surgical procedure; wherein at the time said surgical procedure said method includes the following steps:
(a) inputting an MRI scan of the prostate gland of said subject;
(b) segmenting the MRI scan to produce at least one segmented MRI contour surface of the organ, said contour comprising a plurality of three-dimensional (3D) landmark points;
(c) inputting a TRUS scan of the prostate gland of said subject;
(d) segmenting the TRUS scan to produce at least one segmented TRUS contour surface of the prostate gland, said contour comprising a plurality of 3D landmark points, wherein the at least one MRI contour surface and the at least one TRUS contour surface correspond to the same anatomical surface;
(e) resampling the TRUS and MRI contours to a common geometric space;
(f) computing a linear transformation that maps the MRI contour surface onto the TRUS contour surface, the linear transformation being an affine transformation estimated by minimization of the matching cost between the plurality of landmark points on the MRI contour and the plurality of landmark points on the TRUS contour;
(g) applying said linear transformation to the MRI contour points to obtain linearly transformed (LT) MRI contour points;
(h) computing a local shape descriptor for each LT landmark point of the MRI contour surface and each landmark point of the TRUS contour surface;
(i) computing an optimal assignment between said LT landmark MRI and TRUS contour surface points that minimizes a matching cost criterion between the shape descriptors of the matched points, said optimal assignment defining a sparse vector field mapping MRI contour points onto TRUS contour points;
(j) computing a dense deformation field by smooth interpolation of said sparse vector field to map any point of the whole MRI volume onto a point of the TRUS volume;
and
(k) applying the linear and non-linear mapping of steps (f) through (j) to map points of the MRI image into the TRUS image;
wherein the performance of steps (a) through (k) generates a Trans-Rectal Ultra-Sound-Magnetic Resonance Imaging fusion image of said prostate, and said fusion image is used in targeting an area of the prostate for surgical treatment or diagnosis in said subject having cancer or suspected of having cancer.

25. The method of claim 24, wherein said at least one contour surface comprises an external surface of the prostrate or a portion thereof, a contour of an internal surface of the prostate or a portion thereof, a contour of a transitional zone of the prostate or a portion thereof, a contour of a central zone of the prostate or a portion thereof, a contour of a peripheral zone of the prostate or a portion thereof, a contour of an interface between a central zone and a peripheral zone of the prostate or a portion thereof, a contour of a surface bordering the prostate and the urethra or a portion thereof, a contour based on observable calcifications, or any combinations thereof, or any combination thereof.

26. (canceled)

27. The method of claim 24, wherein said minimizes a matching cost criterion of step (i) is computed according to the count distribution of contour points falling within a plurality of histogram bins neighboring each landmark point.

28. The method of claim 24, wherein the dense deformation field is constrained to be smooth and invertible.

29. (canceled)

30. The method of claim 24, wherein said surgical procedure comprises a focal procedure, wherein said focal procedure comprises a prostatectomy, a robotic prostatectomy, a biopsy, an image guided biopsy, brachytherapy, cryotherapy, a high intensity focalized ultrasound therapy, a vascular targeted photodynamic therapy, a radiotherapy, an external beam radiotherapy, or a surgery for removal of a tumor, or any combination thereof.

31. (canceled)

32. (canceled)

33. The method of claim 24, wherein said target is the complete prostate gland, a region of the prostate gland, a tumor within the prostate gland, or any combination thereof.

34. The method of claim 24, wherein said fused image provides a visualization and localization of the neurovascular bundle adjacent to the prostate gland, wherein said improving the accuracy of determining the location to target during said medical procedure further comprises improving the accuracy of determining a location to avoid during said medical procedure in order that said neurovascular bundle is not damaged.

35.-70. (canceled)
